(19) United States
(12) Patent Application Publication
(10) Pub. No.: US 2005/0124702 A1
(43) Pub. Date: Jun. 9, 2005

(54) Use of organic buffering agents to enhance the antimicrobial activity of pharmaceutical compositions

(75) Inventors: Howard Allen Ketelson, Fort Worth, TX (US); Nissanke L. Dassanayake, Arlington, TX (US); David L. Meadows, Colleyville, TX (US)

Correspondence Address: ALCON RESEARCH, LTD., R&D COUNSEL, Q-148, 6201 SOUTH FREEWAY, FORT WORTH, TX, 76134-2099, US

(73) Assignee: Alcon, Inc.

(21) Appl. No.: 11/000,727

(22) Filed: Dec. 1, 2004

Related U.S. Application Data
(60) Provisional application No. 60/528,281, filed on Dec. 9, 2003.

(51) Int. Cl. A61K 31/13

(52) U.S. Cl. 514/669; 510/112

(57) ABSTRACT

The use of organic buffers to enhance the antimicrobial activity of aqueous pharmaceutical compositions is described. The buffers have tri-hydroxy functional groups and terminal acid groups, and are zwitterionic at physiological pH conditions. The most preferred buffer is tricine. The invention is particularly directed to the use of tricine to enhance the antimicrobial activity of ophthalmic compositions, such as solutions for disinfecting contact lenses and artificial tear compositions.

### CLAIM FOR PRIORITY

This application claims priority from U.S. Patent Application Ser. No. 60/528,281, filed Dec. 9, 2003.

### BACKGROUND OF INVENTION

The present invention is directed to the use of organic buffering agents having tri-hydroxy functional groups and terminal acid groups (e.g., tricine) to enhance the antimicrobial activity of pharmaceutical compositions, particularly aqueous ophthalmic compositions.

Many pharmaceutical compositions are required to be sterile (i.e., free of bacteria, fungi and other pathogenic microorganisms). Examples of such compositions include: solutions and suspensions that are injected into the bodies of humans or other mammals; creams, lotions, solutions or other preparations that are topically applied to wounds, abrasions, burns, rashes, surgical incisions, or other conditions where the skin is not intact; and various types of compositions that are applied either directly to the eye (e.g., artificial tears, irrigating solutions, and drug products), or are applied to devices that will come into contact with the eye (e.g., contact lenses).

The foregoing types of compositions can be manufactured under sterile conditions via procedures that are well known to those skilled in the art. However, once the packaging for the product is opened, such that the composition is exposed to the atmosphere and other sources of potential microbial contamination (e.g., the hands of a human patient), the sterility of the product may be compromised. Such products are typically utilized multiple times by the patient, and are therefore frequently referred to as being of a “multi-dose” nature.

Due to the frequent, repeated exposure of multi-dose products to the risk microbial contamination, it is necessary to employ a means for preventing such contamination from occurring. The means employed may be (1) a chemical agent that prevents the proliferation of microbes in the composition, which is referred to herein as an “antimicrobial preservative”; or (2) a packaging system that prevents or reduces the risk of microbes reaching the pharmaceutical composition within a container.

Ophthalmic compositions generally must include an anti-microbial agent to prevent contamination of the compositions by bacteria, fungi and other microbes. Such compositions may come into contact with the cornea either directly or indirectly. The cornea is particularly sensitive to exogenous chemical agents. Consequently, in order to minimize the potential for harmful effects on the cornea, it is necessary to use anti-microbial agents that are relatively non-toxic to the cornea, and to use such agents at the lowest possible concentrations (i.e., the minimum amounts required in order to perform their anti-microbial functions).

Balancing the anti-microbial efficacy and potential toxicological activity of anti-microbial agents is sometimes difficult to achieve. More specifically, the anti-microbial agent concentration necessary for the preservation of ophthalmic formulations from microbial contamination or for the disinfection of contact lenses may create the potential for toxicological effects on the cornea and/or other ophthalmic tissues. Using lower concentrations of the anti-microbial agents generally helps to reduce the potential for such toxicological effects, but the lower concentrations may be insufficient to achieve the required level of biocidal efficacy (e.g., antimicrobial preservation or disinfection).

The use of an inadequate level of antimicrobial preservation may create the potential for microbial contamination of the compositions and ophthalmic infections resulting from such contaminations. This is also a serious problem, since ophthalmic infections involving pseudomonas aeruginosa or other virulent microorganisms can lead to loss of visual function or even loss of the eye.

Thus, there is a need for a means of enhancing the activity of anti-microbial agents so that very low concentrations of the agents can be utilized without increasing the potential for toxicological effects or subjecting patients to unacceptable risks of microbial contamination and resulting ophthalmic infections.

Compositions for treating contact lenses and other types of ophthalmic compositions are generally formulated as isotonic, buffered solutions. One approach to enhancing the anti-microbial activity of such compositions is to include multi-functional components in the compositions. In addition to performing their primary functions, such as cleaning or wetting contact lens surfaces (e.g., surfactants), buffering the compositions (e.g., borate), or chelating undesirable ions (e.g., EDTA), these multi-functional components also serve to enhance the overall anti-microbial activity of the compositions. For example, ethylenediaminetetraacetic acid and the monosodium, disodium and trisodium salts thereof (collectively referred to herein as “EDTA”) has been widely used for many years in ophthalmic products, particularly products for treating contact lenses. EDTA has been used in such products for various purposes, but particularly for its supplemental anti-microbial activity and as a chelating agent. The inclusion of EDTA in contact lens care products and other ophthalmic compositions enhances the anti-microbial efficacy of chemical preservatives contained in such compositions, particularly the efficacy of those preservatives against gram negative bacteria.

The following publications may be referred to for further background regarding the use of multi-functional components to enhance the antimicrobial activity of ophthalmic compositions:
1. U.S. Pat. No. 5,817,277 (Mowrey-McKee, et al; tromethamine);
2. U.S. Pat. No. 6,503,497 (Chowhan, et al.; borate/polyol complexes);
3. U.S. Pat. No. 5,741,817 (Chowhan, et al.; low molecular weight amino acids such as glycine);
4. U.S. Pat. No. 6,319,464 (Asgharian; low molecular weight amino alcohols); and
5. U.S. Patent Application Publication No. U.S. 2002/0122831 A1 (Mowrey-McKee, et al.; bis-aminopolyols).

The use of tricine as a buffer in ophthalmic compositions is described in the following publications:
1. U.S. Pat. No. 6,162,393 (De Bruiju, et al.);
2. International Publication No. WO 00/71175 A1 (Tuse, et al.); and
3. International Publication No. WO 95/01414 (Vigh).

The present invention is directed to a new approach for enhancing the antimicrobial activity of aqueous pharmaceutical compositions, particularly ophthalmic compositions.

### SUMMARY OF THE INVENTION

The present invention is directed to the use of organic buffers that have tri-hydroxyalkyl functional groups and terminal acid groups to enhance the antimicrobial activity of pharmaceutical compositions. The invention is particularly directed to methods for enhancing the antimicrobial activity of aqueous ophthalmic compositions, such as artificial tear compositions and solutions for disinfecting contact lenses. The most preferred organic buffer is tricine.

The above-described organic buffers are utilized in combination with borate, borax or other boron-containing substances. This combination has been found to enhance the antimicrobial activity of ophthalmic compositions.

The organic buffers described herein may be used in various types of ophthalmic compositions, particularly compositions for treating contact lenses, such as disinfectants, cleaners, comfort drops and rewetting drops, as well as artificial tears, ocular lubricants. The organic buffers are particularly useful in compositions for disinfecting, rinsing, storing and/or cleaning contact lenses. When these compounds are combined with borate or other boron-containing substances, the anti-microbial effect of the organic buffer/borate combination reduces the amount of anti-microbial agent required for preservative purposes, and in some instances, may totally eliminate the need for conventional anti-microbial preservative agents. Multi-dose compositions that do not contain any conventional antimicrobial preservatives (e.g., benzalkonium chloride, chlorhexidine, polyquaternium-1, etc.) are referred to herein as being “preservative free” or “self-preserved”.

The present invention is particularly directed to the provision of improved compositions for disinfecting contact lenses. The compositions exhibit enhanced anti-microbial activity. The enhancement is achieved by means of a combination of formulation criteria, including the use of an organic buffer in combination with a boron-containing compound, as described herein.

### DETAILED DESCRIPTION OF THE INVENTION

The organic buffers utilized in the present invention include two functional moieties: (i) a trihydroxylalkyl moiety; and (ii) a terminal acid moiety, such as carboxylic, sulfonic or phosphonic acid groups. Compounds having terminal carboxylic acid groups are preferred.

The most preferred organic buffer is N-[tris(hydroxymethyl)methyl] glycine, which is also known as “tricine”. The organic buffers utilized in the present invention, such as tricine, have both basic and acidic groups, and as a result are zwitterionic. Under physiological pH conditions, these buffers carry both a positive and a negative charge.

The pka of tricine is 8.15 (D. D. Perrin and B. Dempsey, “Buffers for pH and Metal Ion Control” p. 42, Chapman and Hall, NY (1974)). Its chemical structure and equilibrium states are shown below:

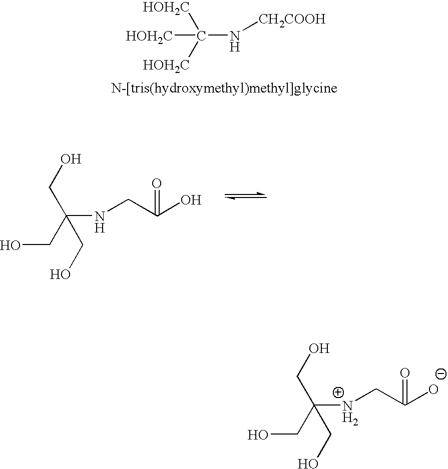

The addition of tricine to ophthalmic formulations in the presence of boric acid has been found to enhance the anti-microbial activity of a biocide, when compared to the same formulations without tricine. Biocides from a variety of classes were tested (i.e., polybiguanides, biguanides, and quaternary ammonium compounds), and in all cases the formulations containing tricine and borate were found to have enhanced microbiological efficacy relative to controls.

The amount of organic buffer utilized will depend on the particular buffer selected, the other ingredients in the composition (i.e., other anti-microbial agents, chelating agents, buffering agents or tonicity agents), and the function of the anti-microbial agents contained in the ophthalmic compositions (i.e., preservation of compositions or disinfection of contact lenses). In general, one or more of the above-described organic buffers will be utilized in a concentration of from about 0.01 to about 2.0 percent by weight/volume (“% w/v”), and preferably from 0.05 to 0.5% w/v.

The levels of antimicrobial activity required to preserve ophthalmic compositions from microbial contamination or to disinfect contact lenses are well known to those skilled in the art, based both on personal experience and official, published standards, such as those set forth in the United States Pharmacopoeia (“USP”) and similar publications in other countries.

The organic buffers described herein may be included in various types of ophthalmic compositions to enhance anti-microbial activity. Examples of such compositions include: ophthalmic pharmaceutical compositions, such as topical compositions used in the treatment of glaucoma, infections, allergies or inflammation; compositions for treating contact lenses, such as cleaning products and products for enhancing the ocular comfort of patients wearing contact lenses; and various other types of compositions, such as ocular lubricating products, artificial tears, astringents, and so on. The compositions may be aqueous or non-aqueous, but will generally be aqueous.

In addition to the organic buffers described above, the compositions of the present invention may contain one or more anti-microbial agents to preserve the compositions from microbial contamination and/or disinfect contact lenses. The invention is not limited relative to the types of antimicrobial agents that may be utilized. The preferred biocides include: polyhexamethylene biguanide polymers (“PHMB”), polyquaternium-1, and the amino biguanides described in co-pending U.S. patent application Ser. No. 09/581,952 and corresponding International (PCT) Publication No. WO 99/32158, the entire contents of which are hereby incorporated in the present specification by reference.

The most preferred amino biguanide is identified in U.S. patent application Ser. No. 09/581,952 as “Compound Number 1”. This compound has the following structure: 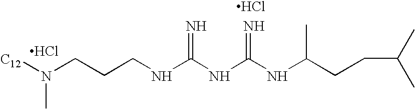
It is referred to below by means of the code number “AL-8496”.

Amidoamines and amino alcohols may also be utilized to enhance the antimicrobial activity of the compositions described herein. The preferred amidoamines are myristamidopropyl dimethylamine (“MAPDA”) and related compounds described in U.S. Pat. No. 5,631,005 (Dassanayake, et al.). The preferred amino alcohols are 2-amino-2-methyl-1-propanol (“AMP”) and other amino alcohols described in U.S. Pat. No. 6,319,464 (Asgharian). The entire contents of the '005 and '464 patents are hereby incorporated in the present specification by reference.

As indicated above, the organic buffers described above are preferably used in combination with borate or borate/polyol buffer systems. As used herein, the term “borate” includes boric acid, salts of boric acid, other pharmaceutically acceptable borates, and combinations thereof. The following borates are particularly preferred: boric acid, sodium borate, potassium borate, calcium borate, magnesium borate, manganese borate, and other such borate salts.

As used herein, the term polyol includes any compound having at least one hydroxyl group on each of two adjacent carbon atoms that are not in trans configuration relative to each other. The polyols can be linear or cyclic, substituted or unsubstituted, or mixtures thereof, so long as the resultant complex is water soluble and pharmaceutically acceptable. Examples of such compounds include: sugars, sugar alcohols, sugar acids and uronic acids. Preferred polyols are sugars, sugar alcohols and sugar acids, including, but not limited to: mannitol, glycerin, xylitol and sorbitol. Especially preferred polyols are mannitol and sorbitol; most preferred is sorbitol.

The use of borate-polyol complexes in ophthalmic compositions is described in U.S. Pat. No. 6,503,497 (Chowhan); the entire contents of which are hereby incorporated in the present specification by reference. The compositions of the present invention preferably contain one or more borates in an amount of from about 0.01 to about 2.0% w/v, more preferably from about 0.05 to 0.5% w/v, and one or more polyols in an amount of from about 0.01 to 5.0% w/v, more preferably from about 0.5 to 2.0% w/v.

The compositions of the present invention may also contain a wide variety of other ingredients, such as tonicity-adjusting agents (e.g., sodium chloride or mannitol), surfactants (e.g., anionic surfactants, such as RLM 100, and nonionic surfactants, such as the poloxamines sold under the name “Tetronic®” and the poloxamers sold under the name “Pluronic®”), and viscosity adjusting agents. The present invention is not limited with respect to the types of ophthalmic compositions in which the organic buffer/borate systems described herein are utilized.

The ophthalmic compositions of the present invention will be formulated so as to be compatible with the eye and/or contact lenses to be treated with the compositions. The ophthalmic compositions intended for direct application to the eye will be formulated so as to have a pH and tonicity which are compatible with the eye. This will normally require a buffer to maintain the pH of the composition at or near physiologic pH (i.e., 7.4) and may require a tonicity agent to bring the osmolality of the composition to a level at or near 210-320 milliosmoles per kilogram (mOsm/kg). The formulation of compositions for disinfecting and/or cleaning contact lenses will involve similar considerations, as well as considerations relating to the physical effect of the compositions on contact lens materials and the potential for binding or absorption of the components of the composition by the lens. The compositions will generally be formulated as sterile aqueous solutions.

The following examples are presented to further illustrate selected embodiments of the present invention.

### EXAMPLE 1

Three pairs of contact lens disinfecting solutions were prepared for evaluation. Each pair consisted of a first solution that contained an organic buffer in accordance with the present invention (i.e., tricine), and a second solution that was identical to the first solution, except for the absence of the organic buffer. The compositions of the solutions are shown in Table 1, below:

TABLE 1

| | Formulation Numbers/Concentrations (% w/v) | | | | | |
|---|---|---|---|---|---|---|
| | A1 | A2 | B1 | B2 | C1 | C2 |
| Component | (9319-3A) | (9319-3B) | (9198-43A) | (9198-43B) | (9319-41A) | (9319-41B) |
| AL 8496 | 0.0001 | 0.0001 | — | — | 0.0003 | 0.0003 |
| Polyquaternium-1 | — | — | 0.0011 | 0.0011 | — | — |
| Sorbitol | — | — | — | — | 0.4 | 0.4 |
| Na Borate | — | — | — | — | 0.2 | 0.2 |
| Na citrate dihydrate | — | — | — | — | 0.6 | 0.6 |
| Boric Acid | 0.6 | 0.6 | 0.6 | 0.6 | — | — |
| Sodium Chloride | 0.32 | 0.32 | 0.32 | 0.32 | — | — |
| Propylene Glycol | 0.5 | 0.5 | 9.5 | 0.5 | 1.0 | 1.0 |
| Tricine | — | 0.2 | — | 0.2 | — | 0.2 |
| Poloxamine 1304 | 0.05 | 0.05 | 0.05 | 0.05 | 0.1 | 0.1 |
| Purified water | q.s. | q.s. | q.s. | q.s. | q.s. | q.s. |
| HCl/NaOH | Adj. | Adj. | Adj. | Adj. | Adj. | Adj. |
| Osmolality | — | — | — | — | — | — |
| PH | 7.8 | 7.8 | 7.0 | 7.0 | 7.8 | 7.8 |

The solutions were prepared as follows: 250 mL beakers were filled with purified water (at room temperature) to 80% of total batch volume and the pre-weighed ingredients for the formulations were added with stirring for 20 minutes. Purified water was added to bring the solutions to 95% of the total batch volume and the pH was measured and adjusted with HCl or NaOH. When the target pH was obtained, the biocides were added to the formulations and the volume brought up to 100% of the batch volume. The pH was measured again and adjusted, if necessary, and the osmolality was recorded.

The antimicrobial activity of the solutions described in Table 1 was evaluated by means of the following procedure:

### General Test Procedure To Screen Antimicrobial Compounds and Experimental Test Formulations

The bacteria Serratia marcescens ATCC 13880 and Staphylococcus aureus ATCC 6538 are cultured on soybean casein digest agar (SCDA) slants. The yeast Candida albicans ATCC 10231 is cultured on Sabouraud Dextrose Agar slants. Surface growth of the three microorganisms is harvested with phosphate buffered saline containing Polysorbate 80. The microbial suspensions are adjusted spectrophotometrically to a concentration of approximately 1.0×10⁸ colony forming units per mL (CFU/mL).

Antimicrobial compounds are prepared initially at target concentrations in selected vehicles, commonly water, a borate buffered saline or other test vehicle. Ten mL of test solution are inoculated with 0.1 mL of the appropriate microbial suspension so that the test solution contains approximately 1.0×10⁶ CFU/mL. The tubes are thoroughly mixed and kept at room temperature during the test.

At six and 24 hours after test solution inoculation, a 1.0 mL aliquot from each test sample and for each challenge organism is transferred to 9.0 mL Dey Engley Neutralizing Broth blanks. The samples are serially diluted in the neutralizing broth and pour plates are prepared from appropriate dilutions with SCDA containing neutralizing agents. Petri plates are incubated for 48-72 hours and the number of survivors visible as discrete colony forming units are determined according to standard microbiological methods.

The results of the evaluation are presented in Table 2, below:

TABLE 2

| | Formulation Numbers/Log Order Reductions | | | | | | |
|---|---|---|---|---|---|---|---|
| | Time | A1 | A2 | B1 | B2 | C1 | C2 |
| Microorganism | (hrs) | (9319-3A) | (9319-3B) | (9198-43A) | (9198-43B) | (9319-41A) | (9319-41B) |
| Candida albicans | 6 | 2.0 | 2.1 | 2.4 | 2.4 | 2.3 | 2.3 |
| | 24 | 3.0 | 2.8 | 3.0 | 3.3 | 4.6 | 4.8 |
| Serratia marcescens | 6 | 2.8 | 2.7 | 6.1 | 4.5 | 3.2 | 4.2 |
| | 24 | 3.0 | 6.0 | 6.1 | 6.0 | 6.0 | 6.0 |
| Staphlococcus | 6 | 3.1 | 2.7 | 3.9 | 6.0 | 5.0 | 4.8 |
| aureus | 24 | 6.1 | 6.1 | 6.0 | 5.0 | 6.0 | 6.0 |

The following conclusions are supported by the microbiological data shown in Table 2:
1. Tricine enhanced the disinfection activity of the formulations across a broad microorganism range.
2. The enhancement of antimicrobial activity was not limited to a particular biocide class.
3. Tricine levels as low as 0.2% were effective in enhancing the antimicrobial activity of the formulations.

### EXAMPLE 2

Table 3 (below) shows three pairs of formulations that were evaluated relative to the effect of tricine on antimicrobial activity levels. Each pair consisted of a first solution containing 1 ppm of the amino biguanide AL-8496 and 2 ppm of the polymeric quaternary ammonium agent polyquaternium-1, and a second solution that was identical to the first solution, except for the inclusion of tricine at a concentration of 0.2% w/v. The formulations were prepared and evaluated via the procedures described in Example 1. The results are presented in Table 3, below:

TABLE 3

| | Formulation Numbers/Concentrations (% w/v) | | | | | |
|---|---|---|---|---|---|---|
| | A1 | A2 | B1 | B2 | C1 | C2 |
| Component | 10363-11A | 10363-11B | 10363-11E | 10363-11F | 10363-11G | 10363-11H |
| Polyquaternium-1 | 0.0002 | 0.0002 | 0.0002 | 0.0002 | 0.0002 | 0.0002 |
| AL-8496A* | 0.0001 | 0.0001 | 0.0001 | 0.0001 | 0.0001 | 0.0001 |
| Sodium borate | 0.6 | 0.6 | 0.6 | 0.6 | 0.6 | 0.6 |
| Poloxamine 1304 | 0.05 | 0.05 | 0.05 | 0.05 | 0.05 | 0.05 |
| Sodium chloride | 0.3 | 0.3 | — | — | — | — |
| Propylene glycol | — | — | 1.0 | 1.0 | 1.0 | 1.0 |
| EDTA | 0.05 | 0.05 | 0.05 | 0.05 | 0.05 | 0.05 |
| Sorbitol | 0.4 | 0.4 | 0.4 | 0.4 | 0.8 | 0.8 |
| Tricine | — | 0.2 | — | 0.2 | — | 0.2 |
| pH | 7.8 | 7.8 | 7.8 | 7.8 | 7.8 | 7.8 |

| | | Formulation Number/Log Order Reductions | | | | | |
|---|---|---|---|---|---|---|---|
| | Time | A1 | A2 | B1 | B2 | C1 | C2 |
| Microorganism | (hrs) | 10363-11A | 10363-11B | 10363-11E | 10363-11F | 10363-11G | 10363-11H |
| C. albicans | 6 | 1.9 | 1.9 | 3.2 | 3.0 | 2.5 | 2.3 |
| 1.1 × 10⁶ | 24 | 3.7 | 3.7 | 5.0 | 4.7 | 4.7 | 4.7 |
| S. marcescens | 6 | 4.0 | 5.4 | 2.8 | 6.1 | 3.1 | 4.2 |
| 1.3 × 10⁶ | 24 | 6.1 | 6.1 | 5.4 | 6.1 | 6.1 | 6.1 |
| S. aureus | 6 | 3.5 | 4.4 | 6.1 | 6.1 | 3.4 | 6.1 |
| 1.2 × 10⁶ | 24 | 6.1 | 6.1 | 6.1 | 6.1 | 4.4 | 6.1 |

*As base

The results demonstrate that there were enhancements of antimicrobial activity when tricine was present in the formulation. For example, a comparison of formulation 11A versus formulation 11B (containing 0.2% tricine) shows that the activity against S. marcescens and S. aureus increased at 6 hours. The log order reductions in the innoculums increased from 4.0 to 5.4 and 3.5 to 4.4 against the bacteria S. marcescens and S. aureus, respectively. The effect of tricine is also evident when comparing formulations 11E and 11F, where the activity against S. marcescens increased from 2.8 to 6.1 at 6 hours, and Formulations 11G and 11H, where the activity against S. marcescens increased from 3.1 to 4.2 and 3.4 to 6.1, at 6 hours and 24 hours, respectively.

1. Use of an organic buffer having tri-hydroxyalkyl functional groups and terminal acid groups to enhance the antimicrobial activity of an aqueous pharmaceutical composition.

2. Use according to claim 1, wherein the composition is an aqueous ophthalmic, otic or nasal composition.

3. Use according to claim 2, wherein the organic buffer comprises tricine.

4. Use according to claim 3, wherein the composition is an ophthalmic composition.

5. Use according to claim 4, wherein the ophthalmic composition is a solution for treating contact lenses.

6. Use according to claim 5, wherein the composition is a contact lens disinfecting solution.

7. Use according to claim 4, wherein the composition is an ocular lubricant or artificial tear.

8. An aqueous ophthalmic, otic or nasal composition comprising an organic buffer having tri-hydroxyalkyl functional groups and terminal acid groups, in an amount effective to enhance the antimicrobial activity of the composition.

9. A composition according to claim 8, wherein the organic buffer comprises tricine.

10. A composition according to claim 9, wherein the composition is an ophthalmic composition.

11. A composition according to claim 10, wherein the composition is a solution for treating contact lenses.

12. A composition according to claim 11, wherein the composition is a contact lens disinfecting solution.

13. A composition according to claim 10, wherein the composition is an ocular lubricant or artificial tear.
